(19) United States
(12) Patent Application Publication
(10) Pub. No.: US 2022/0110829 A1
(43) Pub. Date: Apr. 14, 2022

(54) SYSTEM FOR LYOPHILIZING, RECONSTITUTING, AND DELIVERING A MEDICATION, AND RELATED METHODS

(71) Applicant: Janssen Pharmaceuticals, Inc., Titusville, NJ (US)

(72) Inventors: Ralph Fritz Zwirnmann, Ambler, PA (US); Matthew Piserchio, Chalfont, PA (US); Emma Richard, Philadeelphia, PA (US)

(21) Appl. No.: 17/421,986

(22) PCT Filed: Jan. 10, 2020
(86) PCT No.: PCT/IB2020/050183

§ 371 Date: Jul. 9, 2021

Related U.S. Application Data
(60) Provisional application No. 62/791,182, filed on Jan. 11, 2019.

(51) Int. Cl. A61J 1/20; A61K 39/395; A61K 9/19

(52) U.S. Cl. CPC A61J 1/2096; A61J 1/2048; A61J 1/10; A61K 39/3955; A61K 9/19; A61J 1/2075

(57) ABSTRACT

A modular syringe assembly can include: a tubular syringe barrel having a proximal end and a distal end, the tubular syringe barrel defining a longitudinal axis; a plunger slidable within the tubular syringe barrel; and a cap assembly including a stopper portion and a Luer tip, the stopper portion receivable in the distal end of the tubular syringe barrel, the stopper portion defining a vent window. The cap assembly can be slidable within the tubular syringe barrel along the longitudinal axis between a sealed position where the vent window is covered by the tubular syringe barrel to prevent venting between the cap assembly and the tubular syringe barrel, and a vented position where the vent window forms a vent between the cap assembly and the tubular syringe barrel. Other features and methods are also described.

### CROSS-REFERENCE TO RELATED APPLICATIONS

The present application claims priority to U.S. Provisional Application No. 62/791,182 filed on Jan. 11, 2019, the entire contents of which are expressly incorporated herein by reference.

### TECHNICAL FIELD

The present invention relates to a modular system for mixing two or more substances, such as liquids or powders. More specifically, the present invention relates to a modular system that can be used in lyophilizing, reconstituting, and delivering a medication, and related methods of using the same.

### BACKGROUND

Lyophilized medications are known in the art. Commonly, the lyophilized medication is provided in a vial. To reconstitute the medication, a healthcare provider typically follows a multi-step process that includes: attaching a needle to a syringe; drawing diluent from a vial into the syringe via the needle; and injecting the diluent from the syringe into the vial containing the lyophilized medication, thereby reconstituting the medication. To deliver the medication after reconstitution, the healthcare provider typically withdraws the reconstituted medication from the vial via the syringe with attached needle, and then injects the medication into an IV bag or the patient. This process presents multiple opportunities for the healthcare provider to unintentionally stick themselves or others with the needle. There is a need in the art for systems and methods for lyophilizing, reconstituting, and delivering a medication that overcome these and other disadvantages of the prior art.

### BRIEF SUMMARY

According to an embodiment of the disclosure, a modular syringe assembly can include: a tubular syringe barrel having a proximal end and a distal end, the tubular syringe barrel defining a longitudinal axis; a plunger slidable within the tubular syringe barrel; and a cap assembly including a stopper portion and a Luer tip, the stopper portion receivable in the distal end of the tubular syringe barrel, the stopper portion defining a vent window. The cap assembly can be slidable within the tubular syringe barrel along the longitudinal axis between a sealed position where the vent window is covered by the tubular syringe barrel to prevent venting between the cap assembly and the tubular syringe barrel, and a vented position where the vent window forms a vent between the cap assembly and the tubular syringe barrel.

According to another embodiment, a method of lyophilizing a medication can include: providing a modular syringe assembly containing the medication, the modular syringe assembly including a tubular syringe barrel, a plunger slidable within the tubular syringe barrel, and a cap assembly movable on the tubular syringe barrel between a sealed position and a vented position; lyophilizing the medication with the cap assembly in the vented position, whereby vapor is vented through the cap assembly; and moving the cap assembly from the vented position to the sealed position.

According to yet another embodiment, a method of reconstituting a lyophilized medication can include: providing a modular syringe assembly containing the lyophilized medication, the modular syringe assembly including a tubular syringe barrel, a plunger slidable within the tubular syringe barrel, and a cap assembly including a Luer tip; coupling a plunger rod to the plunger; coupling the Luer tip to a source of diluent; and retracting the plunger rod to draw the diluent into the tubular syringe barrel.

### BRIEF DESCRIPTION OF THE DRAWINGS

FIG. 1 is a side view of an embodiment of a modular syringe assembly;

FIG. 2 is a perspective view of the modular syringe assembly of FIG. 1;

FIG. 3 is an exploded view of the modular syringe assembly of FIG. 1;

FIG. 4 is a side view of a portion of a cap assembly of the modular syringe assembly of FIG. 1;

FIG. 5 is a bottom view of the portion of the cap assembly of FIG. 4;

FIG. 6 is a side, cross-sectional view of the modular syringe assembly of FIG. 1, shown with the cap assembly in a vented position;

FIG. 7 is a side, cross-sectional view of the modular syringe assembly of FIG. 1, shown with the cap assembly in a sealed position;

FIG. 8 is an exploded view of an alternative embodiment of the modular syringe assembly of FIG. 1;

FIG. 9 is an exploded view of another alternative embodiment of the modular syringe assembly of FIG. 1;

FIG. 10 is a perspective view of the modular syringe assembly of FIG. 1 in combination with a compression jig;

FIG. 11 is a side cross-sectional view of the compression jig of FIG. 10;

FIG. 12 is a perspective view of the modular syringe assembly of FIG. 1 with a plunger rod coupled thereto;

FIG. 13 is a flow diagram of an embodiment of a method of lyophilizing a medication;

FIG. 14 is a side view of the modular syringe assembly of FIG. 12 coupled to an IV bag;

FIG. 15 is a side view of the modular syringe assembly of FIG. 12 coupled to a vial via a vial adaptor;

FIG. 16 is a side view of the modular syringe assembly of FIG. 12 coupled to a needle inserted into a vial;

FIG. 17 is a perspective view of the modular syringe assembly of FIG. 12 coupled to a needle;

FIGS. 18A, 18B, and 18C depict perspective, side, and lateral cross-sectional views of an embodiment of a plunger;

FIGS. 19A, 19B, and 19C depict perspective, side, and lateral cross-sectional views of another embodiment of the plunger; and

FIGS. 20A, 20B, and 20C depict perspective, side, and lateral cross-sectional views of yet another embodiment of the plunger.

### DETAILED DESCRIPTION

All publications, including but not limited to patents and patent applications, cited in this specification are herein incorporated by reference as though fully set forth.

As used herein and in the claims, the singular forms “a,” “and,” and “the” include plural reference unless the context clearly dictates otherwise.

The present application relates to a modular syringe assembly that can be used to combine two or more substances, such as liquids or powders. In a specific implementation, the modular syringe assembly can be used with a lyophilized medication. The modular syringe assembly can be transformed among a variety of different configurations that can permit the modular syringe assembly to be used during the lyophilization, reconstitution, and/or delivery of the medication. By utilizing a modular syringe assembly during some or all of the foregoing steps, the risk of needle sticks associated with conventional processing of lyophilized medications (which often use a needle to transfer the medication between various containers) can be reduced or eliminated.

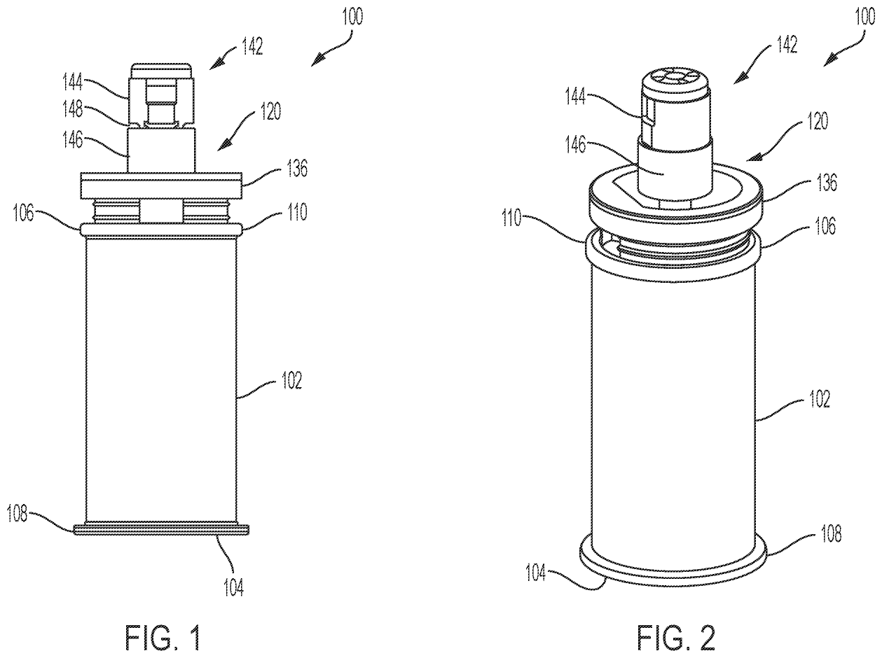

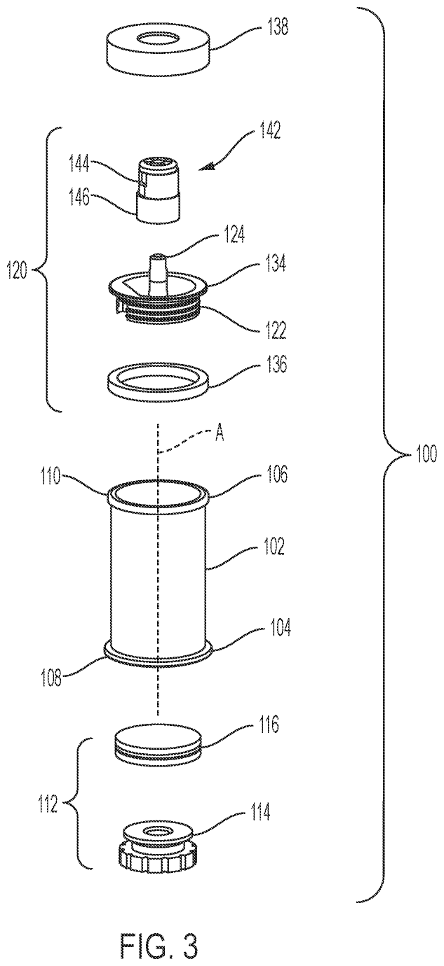

Referring to FIGS. 1-3, an embodiment of a modular syringe assembly 100 is shown. FIG. 1 is a side view of the modular syringe assembly 100. FIG. 2 is a perspective view of the modular syringe assembly 100. FIG. 3 is an exploded view of the modular syringe assembly 100. The modular syringe assembly 100 can include a tubular syringe barrel 102. The tubular syringe barrel 102 can define a longitudinal axis A, shown in FIG. 3. The tubular syringe barrel 102 can define a hollow interior portion for containing the medication, and can include a proximal end 104 and a distal end 106, both of which are open. A protruding lip 108 can be provided at the proximal end 104. The protruding lip 108 can facilitate attachment of a finger grip to the tubular syringe barrel 102. A protruding lip 110 can be provided at the distal end 106. The tubular syringe barrel 102 can be made of glass, plastic, or other suitably durable materials known in the art. For example, and without limitation, the tubular syringe barrel 102 can be made of borosilicate glass, polypropylene, crystal zenith, Makrolon RX, or polycarbonate.

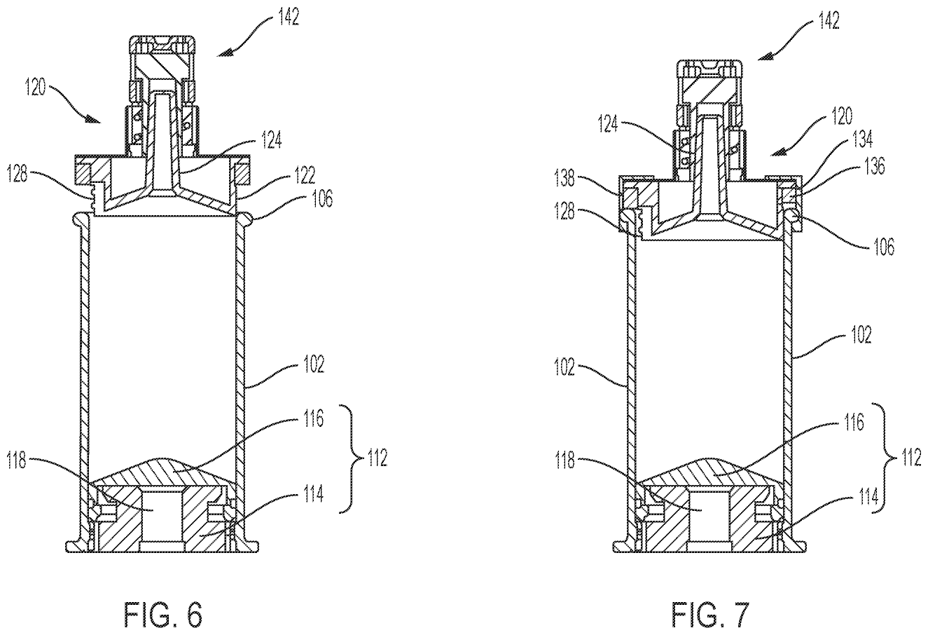

Referring to FIG. 3, the modular syringe assembly 100 can include a plunger 112 that is slidable within the tubular syringe barrel 102. In the embodiment of FIG. 3, the plunger 112 comprises a rigid plunger base 114 having a rubber plunger tip 116 coupled thereto, however, in alternative embodiments, the rigid plunger base 114 can be omitted and the plunger 112 can be formed entirely of a pliable material such as rubber. The plunger 112 can slide within and form a seal to the interior of the tubular syringe barrel 102. Referring to FIGS. 6 and 7, embodiments of the plunger 112 can include a coupling 118 for connection to a removable plunger rod (not shown). For example, the coupling 118 can comprise a female thread or quarter-turn cam configured to receive a male counterpart on the plunger rod, or vice versa. According to embodiments, and without limitation, the rigid plunger base 114 can be formed of polypropylene, crystal zenith, Makrolon RX, or polycarbonate. According to embodiments, and without limitation, the plunger tip 116 can be formed of bromobutyl rubber.

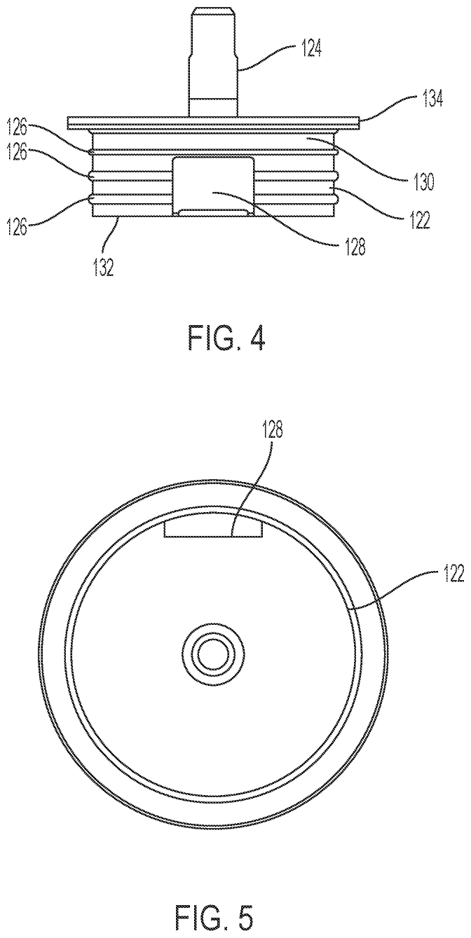

Referring back to FIGS. 1-5, the modular syringe assembly 100 can further include a cap assembly 120 that attaches to the distal end 106 of the tubular syringe barrel 102. Referring to FIGS. 4 and 5, the cap assembly 120 can include a stopper portion 122 and a Luer tip 124. The stopper portion 122 and Luer tip 124 can be co-molded parts, or alternatively, can be separate parts joined together, for example, by bonding. The stopper portion 122 can have an outer diameter that allows it to slide within the distal end 106 of the tubular syringe barrel 102, while forming a seal with the tubular syringe barrel 102. As shown in FIG. 4, the stopper portion 122 can include ribs 126 or other surface features to form a seal with the tubular syringe barrel 102, yet provide for a sliding interface therewith. As best seen in FIGS. 4 and 5, the stopper portion 122 can define a vent window 128.

Referring to FIGS. 6 and 7, the modular syringe assembly is shown in longitudinal cross-section. The cap assembly 120 is slidable with respect to the tubular syringe barrel 102 along the longitudinal axis A (FIG. 3) between a vented position shown in FIG. 6 and a sealed position shown in FIG. 7. In the vented position of FIG. 6, the cap assembly 120 is raised up from the distal end 106 of the tubular syringe barrel 102. In this position, the vent window 128 forms a vent passage between the stopper portion 122 and the tubular syringe barrel 102, allowing vapor to escape from within the tubular syringe barrel 102 into the ambient. In the sealed position of FIG. 7, the vent window 128 is covered by the tubular syringe barrel 102, thereby blocking airflow between the tubular syringe barrel 102 and the cap assembly 120 (and obstructing venting between the interior of the tubular syringe barrel 102 and the ambient). With the cap assembly 120 in the sealed position and the plunger 112 in the tubular syringe barrel 102, the space between the cap assembly 120 and the plunger 112 is sealed. A medication, such as lyophilized medication, can be located in the tubular syringe barrel 102 in the sealed space between the cap assembly 120 and plunger 112, as will be described in more detail below. An example of a lyophilized medication can include infliximab.

Referring specifically to FIG. 4, the stopper portion 122 includes a distal end 130 and a proximal end 132 opposed to the distal end 130. The distal end 130 can define a flange 134, for example, that projects radially from the stopper portion 122. According to embodiments, the vent window 128 can be spaced apart from the distal end 130, and can intersect with the proximal end 132. As best seen in FIG. 3, a seal ring 136, such as a gasket, can be located around the stopper portion 122. When the cap assembly 120 is in the sealed position of FIG. 7, the seal ring 136 can be sandwiched between the flange 134 and the distal end 106 of the tubular syringe barrel 102, as shown in FIG. 7. When the cap assembly 120 is in the vented position of FIG. 6, the cap assembly 120 can be raised up such that the flange 134 is spaced apart from the seal ring 136 and/or the seal ring 136 is spaced apart from the distal end 106 of the tubular syringe barrel 102. According to embodiments omitting the seal ring 136, the flange 134 can be spaced apart from the distal end 106 of the tubular syringe barrel 102 when in the vented position, and the flange 134 can contact the distal end 106 of the tubular syringe barrel 102 when in the sealed position. Referring to FIGS. 3 and 7, a crimp ring 138 can be placed over the flange 134 to retain the cap assembly 120 in the sealed position. The protruding lip 110 on the distal end 106 of the tubular syringe barrel 102 can aid in retention of the crimp ring 138.

Referring to FIGS. 1-3, the cap assembly 120 can further include a Luer tip 124. According to embodiments, the Luer tip 124 can be co-molded with the stopper portion 122 (see FIG. 6), however, other embodiments are possible. For example, the Luer tip 124 and the stopper portion 122 can comprise a molded plastic part, however, other embodiments are possible as will be described below. According to embodiments, and without limitation, the Luer tip 124 and/or stopper portion 122 can be formed of polypropylene, crystal zenith, Makrolon RX, or polycarbonate.

Still referring to FIGS. 1-3, an OVS closure 142 (e.g., manufactured by Vetter Pharma of Ravensburg, Germany) can be provided on the Luer tip 124. The OVS closure 142 can snap onto the Luer tip 124. The OVS closure 142 can include a primary closure 144 and a threaded portionl 146 joined by a frangible, tamper-evident seal 148 (see FIG. 1). In use, the tamper-evident seal 148 can be broken by a user to allow removal of the primary closure 144 and thereby expose the Luer tip 124 and the threaded portion 146 of the OVS closure 142. As will be described in more detail below, another component can be coupled to the Luer tip 124 and threaded portion 146 for dispensing of the contents of the modular syringe assembly 100. For example, an EZ-Fill® Integrated Tip Cap from Ompi, Stevanato Group of Newtown, Pa., USA can be used.

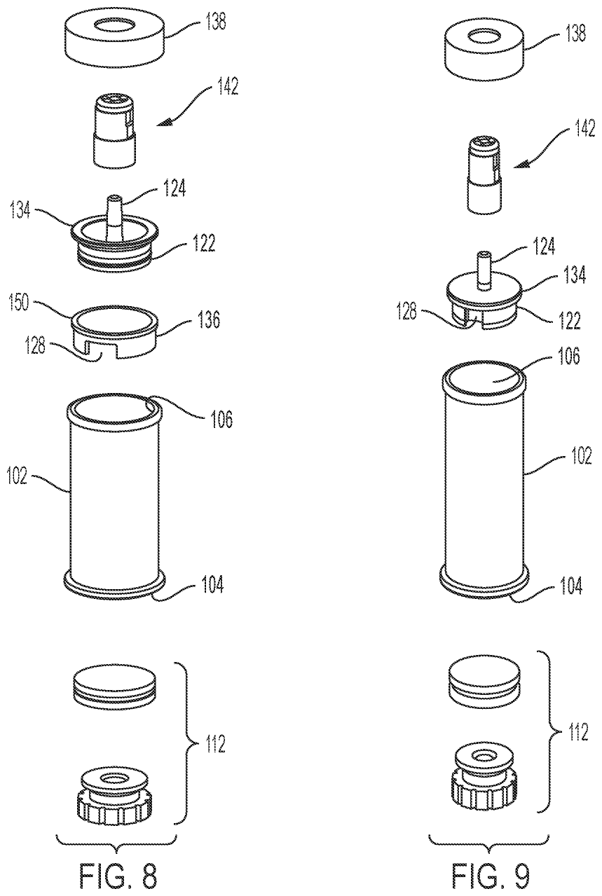

FIG. 8 is an exploded view of an alternative embodiment of the modular syringe assembly 100. The embodiment of FIG. 8 is the same as FIGS. 1-7 except for the differences described below. According to the embodiment of FIG. 8, the sealing ring 136 can fit radially between stopper portion 122 and the inner wall of the distal end 106 of the tubular syringe barrel 102, forming a seal between. In this embodiment, the sealing ring 136 can also include a flange 150 that is sandwiched between the distal end 106 of the tubular syringe barrel 102 and the flange 134 on the stopper portion 122 when the cap assembly 120 is in the sealed position. Additionally, the vent window 128 can be located in the sealing ring 136. Accordingly, the sealing ring 136 can be moved upwards and downwards with respect to the distal end 106 of the tubular syringe barrel 102 to adjust whether the cap assembly is in the sealed or vented position. According to the embodiment of FIG. 8, the stopper portion 122 can be a relatively rigid plastic item, and the sealing ring 136 can comprise a relatively flexible rubber or plastic item that promotes sealing between the stopper portion 122 and the tubular syringe barrel 102.

FIG. 9 is an exploded view of yet another alternative embodiment of the modular syringe assembly. The embodiment of FIG. 9 is the same as FIGS. 1-7 except for the differences described below. In the embodiment of FIG. 9, the seal ring can be omitted. Additionally or alternatively, the stopper portion 122, flange 134, and Luer tip 124 can be co-molded from a rubber material. As shown, the vent window 128 can be co-molded into the stopper portion 122. According to embodiments, and without limitation, the stopper portion 122, flange 134, and/or the Luer tip 124 can be co-molded: the stopper and flange from bromobutyl rubber, the Luer tip from polypropylene, Crystal zenith, Makrolon RX, or Polycarbonate.

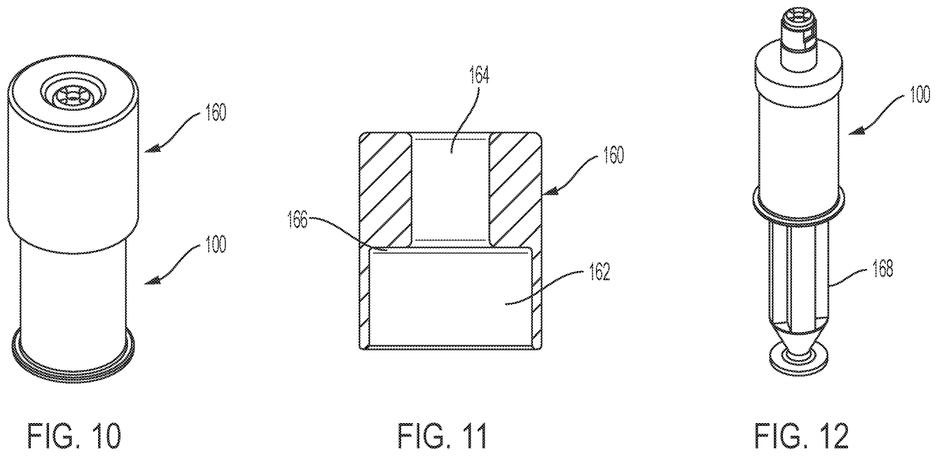

FIG. 10 is a perspective view of the modular syringe assembly 100 of FIG. 1 in combination with a compression jig 160. FIG. 11 is a side cross-sectional view of the compression jig 160 of FIG. 10. The compression jig 160 can be used to apply pressure on the cap assembly 120 to press it into the distal end 106 of the tubular syringe barrel 102, for example, to move the cap assembly 120 from the vented position to the sealed position. The pressure can be applied to the compression jig 160 by a machine surface, such as, for example, a lyophilization shelf or other movable surface. The compression jig 160 can have a geometry that mates with the cap assembly 120 in a manner that eliminates pressure upon the OVS closure 142, reducing the chances of the OVS closure 142 or parts of the cap assembly 120 from becoming damaged as the cap assembly 120 is moved into the sealed position.

As shown in FIG. 11, for example, the compression jig 160 can include a first cavity 162 and a second cavity 164 separated by a contact surface 166. The first cavity 162 can define an inner diameter that permits the first cavity 162 to pass over the cap assembly 120 and distal end 106 of the tubular syringe barrel 102. According to embodiments, the inner diameter of the first cavity 162 can also provide a clearance for vapor from the vent window 128 to pass between the compression jig 160 and the modular syringe assembly 100. The second cavity 164 can define an inner diameter that permits it to pass over the OVS closure 142. The contact surface 166 can comprise a substantially traverse surface that rests on the upper surface of the cap assembly 120 to apply pressure from the compression jig 160 onto the cap assembly 120. According to an embodiment, the inner diameter of the second cavity 164 can be smaller than the inner diameter of the first cavity 162. In use, the compression jig 160 can apply pressure onto the flange 134 via contact surface 166 of the cap assembly 120 to move the cap assembly 120 into the distal end 106 of the tubular syringe barrel 102, e.g., to move the cap assembly 120 from the vented position to the sealed position. The second cavity 164 in the compression jig 160 can serve as a relief for the OVS closure 142, thereby reducing or eliminating contact between the compression jig 160 and the OVS closure 142. This can help reduce or eliminate the potential for the OVS closure 142 and/or Luer tip 124 to be damaged as the cap assembly 120 is moved into the sealed position. Although the contact surface 166 is shown in FIG. 11 as being substantially perpendicular to the axis of the first cavity 162 and the axis of the second cavity 164, other configurations are possible provided the contact surface 166 can transmit sufficient force to the cap assembly 120 to press it into the distal end 106 of the tubular syringe barrel 102.

FIG. 12 is a perspective view of the modular syringe assembly 100 of FIG. 1 with a plunger rod 168 coupled thereto, e.g., removably coupled to the plunger 112.

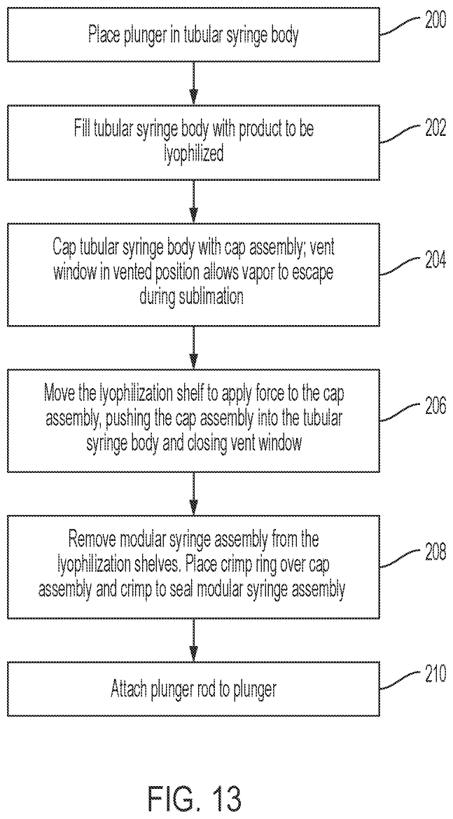

FIG. 13 is a flow diagram of an embodiment of a method of lyophilizing a medication. The method can be performed with the embodiments of the modular syringe assembly 100 described herein. In step 200, the plunger 112 can be inserted into the tubular syringe barrel 102, for example, into the proximal end 104. The location of the plunger 112 within the tubular syringe barrel 102 can be varied to account for different volumes of medication to be lyophilized. For larger volumes, the plunger 112 can be located toward the proximal end 104, whereas for smaller volumes, the plunger 112 can be spaced upward from the proximal end 104.

Next, in step 202, the tubular syringe barrel 102 can be filled with the medication to be lyophilized, for example, using conventional laboratory equipment. An example of a medication that can be lyophilized in the process is infliximab. According to an embodiment, the proximal end 104 of the tubular syringe barrel 102 can rest on the cooling shelf of a lyophilization line during the filling step.

In step 204, the cap assembly 120 is inserted into the distal end 106 of the tubular syringe barrel 102 and located in the vented position. According to various embodiments, step 200 can be performed before or after step 204, provided either the plunger 112 or the cap assembly 120 is applied to the tubular syringe barrel 102 before the medication is added. Once the medication is contained in the tubular syringe barrel, it can be lyophilized using conventional lyophilization equipment. According to an embodiment, the lyophilization process can take less than about 72 hours. With the cap assembly 120 in the vented position, vapor released during the lyophilization process can escape through the open vent window 128 in the cap assembly 120.

In step 206, the cap assembly 120 is moved from the vented position to the sealed position. For example, the cap assembly 120 can be pressed into the distal end 106 of the tubular syringe barrel 102 until the flange 134 contacts the distal end 106 of the tubular syringe barrel 102. Alternatively, for embodiments including a seal ring 136, the cap assembly 120 can be pressed until the seal ring 136 is sandwiched between the flange 134 and the distal end 106 of the tubular syringe barrel 102. In either case, when the cap assembly 120 is moved to the sealed position, the tubular syringe barrel 102 seals off the vent window 128, e.g., obstructs passage of air between the interior of the tubular syringe barrel 102 and the ambient via the vent window 128. An induction foil seal can be applied to the proximal end 104 to further seal that end of the tubular syringe barrel 102.

Step 206 can be performed by applying force to the cap assembly 120 while holding the tubular syringe barrel 102 in place, thereby displacing the cap assembly 120 into the distal end 106 of the tubular syringe barrel 102. This can be done, for example, by pressing on the cap assembly 120. According to embodiments, this can be performed by applying force to the cap assembly 120 using a conventional lyophilization shelf, however, other surfaces or structures can alternatively be used. According to embodiments, force can be transmitted from the lyophilization shelf to the cap assembly 120 via compression jig 160 located between the lyophilization shelf and cap assembly 120.

In step 208, the modular syringe assembly can be removed from the lyophilization shelf and a crimp ring 138 can be secured over the cap assembly 120 to seal the modular syringe assembly 100 and the lyophilized medication contained therein. This can be performed using conventional equipment.

Although the foregoing method was described above in connection with a single modular syringe assembly 100, the same method can be performed to simultaneously batch process multiple modular syringe assemblies and their contained medications.

Still referring to FIG. 13, in step 210 a plunger rod 168 can be coupled to the plunger 112 to facilitate reconstitution and/or delivery of the lyophilized medication. For example, the plunger rod 168 can be coupled to the plunger 112 by securing a threaded connection, cam-lock, or other connection between the plunger rod 168 and plunger 112.

Embodiments of the present invention also include methods of reconstituting and/or delivering a lyophilized medication. To reconstitute the medication, the primary closure 144 can be separated from the OVS closure 142 via frangible seal 148 to reveal the Luer tip 124 of the cap assembly 120. The threaded portion 146 of the OVS closure 142 can remain snapped onto the Luer tip 124, and can be utilized to attach a source of diluent, such as a water vial, to the Luer tip 124. The plunger rod 168 can then be retracted to draw the diluent into the tubular syringe barrel 102 to thereby reconstitute the lyophilized medication. The source of diluent can be decoupled from the Luer tip, for example, by untwisting from the threaded portion 146. According to embodiments, and without limitation, the diluent can include water for injection (WFI), saline, or a liquid custom formulated for the drug product. According to embodiments, and without limitation, the source of diluent can comprise a glass or plastic vial, bottle, or bag.

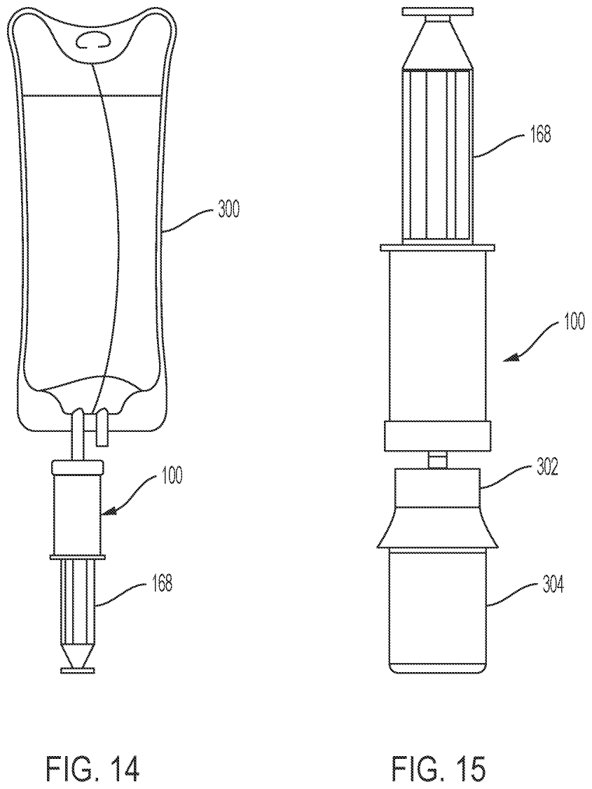

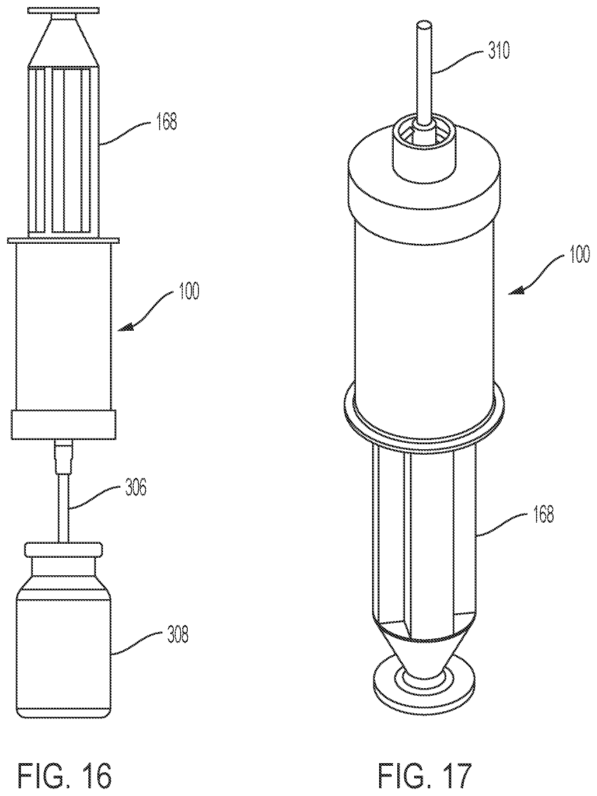

After reconstitution, the medication can be delivered via the modular syringe assembly 100, for example, by pressing the plunger rod 168. With reference to FIGS. 14-17, the Luer tip can be attached, without limitation, to an IV bag 300 (FIG. 14), a vial adaptor 302 attached to a vial 304 (FIG. 15), a needle 306 inserted into a vial 308 (FIG. 16), or a needle 310 (FIG. 17). Although not shown, according to other embodiments, one or more of the modular syringe assemblies 100 can be placed in an automated syringe pump or a customized device such as an autoinjector to dispense the reconstituted medicine. The devices can then be used to administer the reconstituted medication to a patient using techniques known in the art.

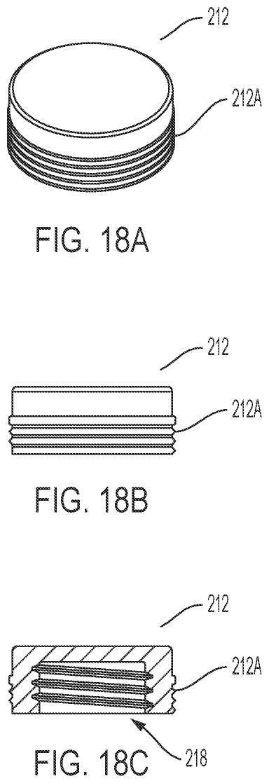

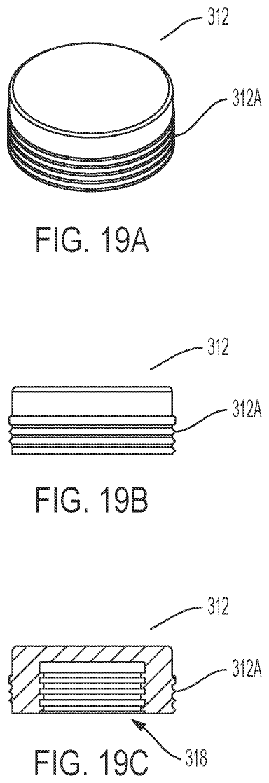

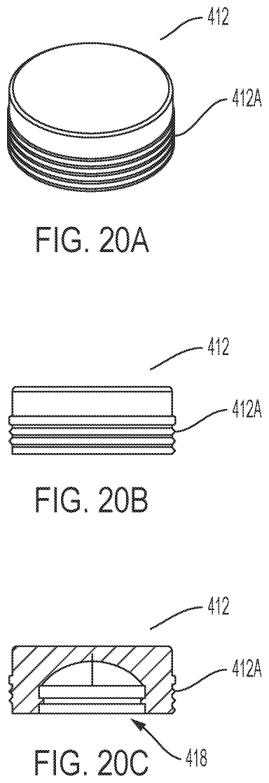

FIGS. 18A-C, 19A-C, and 20A-C depict alternate embodiments of the plunger 112 that can connect directly to a plunger rod, e.g., without requiring the rigid plunger base 114 of FIG. 3. In the embodiment of FIGS. 18A-C, the plunger 212 includes a coupling 218 in the form of a cavity including threads adapted to engage with corresponding threads on the distal end of a plunger rod. In the embodiment of FIGS. 19A-C, the plunger 312 includes a coupling 318 in the form of a cavity including a plurality of ribs adapted to engage with corresponding ribs on the distal end of a plunger rod. The plunger 412 of FIGS. 20A-C also includes a coupling 418 in the form of a ribbed cavity including a rib to engage with ribs on the distal end of a plunger rod. The coupling 318 of FIGS. 19A-C includes a substantially flat top portion to receive a plunger rod having a corresponding flat top. The coupling 418 of FIGS. 20A-C includes a substantially dome-shaped top portion to receive a plunger rod having a corresponding dome-shaped top.

The rubber plungers 212, 312, 412 can also include a plurality of ribs 212A, 312A, 412A, respectively, adapted to form a seal between the plunger and the tubular syringe barrel 102. According to embodiments, and without limitation, the plungers 212, 312, 412 can be formed of bromobutyl rubber.

According to alternate embodiments, various components of the modular syringe assembly 100 can be colored, tinted, and/or solid in color to protect light sensitive drug products. According to further alternate embodiments, the modular syringe assembly 100 can be used to combine two liquids or a powder and liquid, or drug coated microspheres and liquid. According to embodiments utilizing powder and microspheres, the powder and microspheres can filled into the device with the liquid added later as with a lyophilized drug, although alternative sequences are possible depending upon existing process lines. According to still further alternate embodiments, two of the modular syringe assemblies 100 can be joined end to end with a male/male adapter and used to transfer liquid from one to the other as in the case of pre-filled syringes.

What is claimed is:

1. A modular syringe assembly comprising:
a tubular syringe barrel having a proximal end and a distal end, the tubular syringe barrel defining a longitudinal axis;
a plunger slidable within the tubular syringe barrel; and
a cap assembly including a stopper portion and a Luer tip, the stopper portion receivable in the distal end of the tubular syringe barrel, the stopper portion defining a vent window;
wherein the cap assembly is slidable within the tubular syringe barrel along the longitudinal axis between a sealed position where the vent window is covered by the tubular syringe barrel to prevent venting between the cap assembly and the tubular syringe barrel, and a vented position where the vent window forms a vent between the cap assembly and the tubular syringe barrel.

2. The modular syringe assembly of claim 1, wherein the stopper portion further includes a distal end defining a flange, and a proximal end opposed to the distal end, wherein the vent window intersects the proximal end of the stopper portion.

3. The modular syringe assembly of claim 1, wherein the flange contacts the distal end of the tubular syringe barrel when the cap assembly is in the sealed position, and the flange is spaced from the distal end of the tubular syringe barrel when the cap assembly is in the vented position.

4. The modular syringe assembly of claim 1, wherein the cap assembly comprises a sealing ring that fits between the stopper portion and the distal end of the tubular syringe barrel.

5. The modular syringe assembly of claim 4, wherein the vent window is located in the sealing ring.

6. The modular syringe assembly of claim 1, wherein the vent window is molded into the stopper portion.

7. The modular syringe assembly of claim 1, further comprising an OVS closure located on the Luer tip.

8. The modular syringe assembly of claim 1, wherein the plunger includes a coupling for removable attachment to a plunger rod.

9. The modular syringe assembly of claim 8, further comprising a plunger rod removably coupled to the plunger via the coupling.

10. The modular syringe assembly of claim 1, further comprising a lyophilized medication located within the tubular syringe barrel.

11. The modular syringe assembly of claim 10, wherein the lyophilized medication comprises infliximab.

12. A method of lyophilizing a medication, comprising:
providing a modular syringe assembly containing the medication, the modular syringe assembly including a tubular syringe barrel, a plunger slidable within the tubular syringe barrel, and a cap assembly movable on the tubular syringe barrel between a sealed position and a vented position;
lyophilizing the medication with the cap assembly in the vented position, whereby vapor is vented through the cap assembly; and
moving the cap assembly from the vented position to the sealed position.

13. The method of claim 12, further comprising:
inserting the plunger into a proximal end of the tubular syringe barrel;
filling the tubular syringe barrel with the medication; and
inserting the cap assembly into a distal end of the tubular syringe barrel, with the cap assembly located in the vented position.

14. The method of claim 12, wherein moving the cap assembly from the vented position to the sealed position comprises pressing the cap assembly into the distal end of the tubular syringe barrel.

15. The method of claim 14, wherein pressing the cap assembly into the distal end of the tubular syringe barrel seals off a vent window located in the cap assembly.

16. The method of claim 12, wherein moving the cap assembly from the vented position to the sealed position comprises applying force to the cap assembly using a lyophilization shelf.

17. The method of claim 16, wherein applying force to the cap assembly using the lyophilization shelf comprises applying force to a compression jig located between the lyophilization shelf and the cap assembly.

18. The method of claim 12, further comprising applying a crimp ring over the cap assembly when located in the sealed position.

19. The method of claim 12, further comprising attaching a plunger rod to the plunger.

20. The method of claim 12, wherein the medication comprises infliximab.

21. A method of reconstituting a lyophilized medication, comprising:
providing a modular syringe assembly containing the lyophilized medication, the modular syringe assembly including a tubular syringe barrel, a plunger slidable within the tubular syringe barrel, and a cap assembly including a Luer tip;
coupling a plunger rod to the plunger;
coupling the Luer tip to a source of diluent; and
retracting the plunger rod to draw the diluent into the tubular syringe barrel.

22. The method of claim 21, further comprising delivering the reconstituted medication by:
decoupling the source of diluent from the Luer tip; and
depressing the plunger to expel the lyophilized medication through the Luer tip.

23. The method of claim 22, wherein delivering the reconstituted medication further comprises:
coupling at least one of a needle, vial, vial adaptor, or IV bag to the Luer tip.

24. The method of claim 21, further comprising removing a primary closure portion of an OVS closure to expose the Luer tip.

25. The method of claim 21, wherein the lyophilized medication comprises infliximab.
